(19) United States
(12) Patent Application Publication
(10) Pub. No.: US 2005/0118667 A1
(43) Pub. Date: Jun. 2, 2005

(54) Method for detecting microorganisms

(76) Inventors: Alain Rambach, Paris (FR); Alain Le Coustumier, Cahors (FR)

Correspondence Address: FOLEY AND LARDNER;SUITE 500, 3000 K STREET NW, WASHINGTON, DC, 20007, US

(21) Appl. No.: 10/498,245

(22) PCT Filed: Dec. 12, 2002
(86) PCT No.: PCT/FR02/04310

(30) Foreign Application Priority Data
Dec. 13, 2001 (FR) 0116106

(51) Int. Cl. C12Q 1/02; C12Q 1/22; C12Q 1/04

(52) U.S. Cl. 435/29; 435/34; 435/31

(57) ABSTRACT

The invention concerns a method for detecting microorganisms present in a sample, which consists in inoculating a culture medium with said sample, said culture medium preferably containing chromogens releasing chromophores in the presence of target microorganisms or fluorogens, said culture medium being in a container with a coloured base, detection being facilitated by contrast between the coloured base and the isolated colonies.

The present invention relates to a method for detecting microorganisms present in a sample, in which a culture medium is inoculated with said sample, said culture medium preferably containing chromogenic agents releasing chromophores in the presence of target microorganisms or fluorogenic agents, said culture medium being in a container having a colored bottom, the detection being facilitated by the contrast between the colored bottom of the container and the colonies isolated.

Rapid detection of microorganisms present in biological or food samples, or in a hospital environment is essential in order to define as rapidly as possible the measures to be taken in the case of contamination.

It also seems important to be able to differentiate the various contaminants in order to be able to carry out appropriate disinfection.

Methods exist, such as PCR, which is rapid and specific, but which requires the use of apparatus such as thermocyclers, and which cannot always be used in a hospital environment. Moreover, this method may require the preparation of the samples to be tested, which is not always easy to carry out.

Methods based on the culture of the contaminating microorganisms on appropriate media, and on their identification by virtue of the addition of chromogenic agents releasing a chromophore after the action of an enzyme specific to the microorganism have been described.

Thus, patent applications WO 00/53799, WO 00/46345, WO 97/39103, WO 95/04156, WO 94/09152 describe culture media for identifying staphylococci, anaerobic bacteria or enterohemorrhagic coliforms, salmonellae or other microorganisms such as candida.

However, the media used generally being relatively transparent, it can sometimes prove difficult to clearly determine the colors of the chromophores released, because of the lack of sufficient contrast. Thus, the use of the C medium described in patent application WO 95/04156 allows good differentiation of the various candida yeasts, but the colors obtained are light, which may be problematic for some users.

Likewise, the method described in application WO 00/46345 for identifying staphylococci may require adding a contrast agent to the culture medium, for example an opacifier.

Such a need for contrast is also felt for media for detecting salmonellae, for example Rambach Agar sold by the company Chromagar (Paris, France).

There has been proposed in particular the addition of kaolin in order to opacify media, in particular for detecting E. coli, with the aid of phenylglucuronide. However, the addition of this type of component, in addition to increasing the costs of manufacturing the medium, can turn out to pose many problems for the industrial manufacture of detection media (in particular precipitation).

The present invention proposes to respond to the problem posed by the lack of contrast observed between the colonies isolated and the culture medium, in particular when these colonies are colored as a result of the presence of chromogenic agents in the culture medium.

Thus, the present invention relates to a method for detecting microorganisms present in a sample comprising the steps consisting in:
a) preparing a culture medium suitable for said target microorganisms,
b) pouring said medium into a container having a colored bottom,
c) inoculating the solid culture medium from a) with said sample or an inoculum derived from the sample and incubating,
d) detecting the presence of said microorganisms on said culture medium using the contrast between the color of the bottom of the container and the colonies isolated.

In a preferred embodiment, the invention relates to a method for detecting microorganisms present in a sample by enzymatic release of chromophores from chromogenic agents present in a culture medium suitable for said microorganisms, comprising the steps consisting in:
a) preparing an appropriate culture medium containing the chromogenic agents appropriate for the detection of the target microorganisms,
b) pouring said medium into a container having a colored bottom,
c) inoculating the solid culture medium from a) with said sample or an inoculum derived from the sample, and incubating,
d) detecting the presence of the microorganisms present in said sample, on said culture medium, using the contrast linked to the color of the bottom of the container in relation to the color due to the chromophores released.

In another embodiment, the culture medium contains products giving it a color (for example, McConkey Agar and the like).

In another embodiment, the culture medium contains fluorogenic agents, releasing fluorophores in the presence of the target microorganisms. It is possible to use in particular 4-methylumbelliferyl as fluorophore, for example linked to a galactoside (4-methylumbelliferyl-β-D-galactoside).

In a preferred embodiment, the container according to the invention has a round shape and is in particular a Petri dish, having a diameter ranging from 40 mm to 210 mm. Preferably, the diameter of the container according to the invention is equal to 47 mm, 55 mm, 90 mm, 100 mm or 200 mm.

In a preferred embodiment of the invention, the surface of the container is such that it is possible to inoculate the culture medium contained in the container such that growth of the colonies isolated is obtained. Thus, the surface area of the container according to the invention is preferably greater than about 15-17 cm².

In one embodiment of the present invention, said container has an opaque colored bottom. In another embodiment, said container has a transparent colored bottom. Preferably, said container has a bottom having a color chosen from the colors blue, red, yellow, black or opaque white.

In one embodiment of the invention, the said chromophores or other colored products are released by hydrolysis by virtue of the action of an enzyme specific to the microorganism on the chromogenic agents present in the culture medium, and said chromogenic agent(s) is (are) chosen from the enzyme substrates linked to the metabolism of sugars, and in particular from the group consisting of β-glucosidase substrates, β-galactosidase substrates and β-glucuronisase substrates and phosphatase substrates.

Preferably, said chromophore(s) is (are) chosen from the group consisting of indoxyl, haloindoxyl (bromoindoxyl, chloroindoxyl, fluoroindoxyl, iodoindoxyl, dichoroindoxyl, chlorobromoindoxyl, trichloroindoxyl), methylindoxyl and hydroxyquinoline derivatives, in particular from the following derivatives: 6-chloroindoxyl, 5-bromoindoxyl, 3-bromoindoxyl, 6-fluoroindoxyl, 5-iodoindoxyl, 4,6-dichloroindoxyl, 6,7-dichloroindoxyl, 5-bromo-4-chloroindoxyl, 5-bromo-6-chloroindoxyl, 4,6,7-trichloroindoxyl, N-methylindoxyl or 8-hydroxyquinoline.

In order to be able to limit the growth of the microorganisms which are not being searched for, it is advantageous for the culture medium to also contain factors selective for the microorganisms being searched for.

In a particular embodiment, the culture medium also contains a high concentration of sucrose or of glucose.

The method of the invention is advantageously carried out when the target microorganism is of the Candida species, in particular with the Chromagar Candida described in WO 95/04156. A colored, transparent or opaque container is then used, the color depending on the nature of the yeast being the most searched for.

The method of the invention is also particularly suitable when said target microorganism is of the Staphylococcus species and said container is then preferably chosen so that it has an opaque, preferably an opaque white, colored bottom.

It is also possible to carry out the method when said target microorganism is of the Vibrio species, of the species comprising coliforms and in particular enterohemorrhagic bacteria E. coli.

Use is made in particular of the teachings of patent applications WO 00/53799, WO 00/46345, WO 97/39103, WO 95/04156, WO/09152 in order to define the colors of the microorganisms according to the nature of the media obtained. The teachings of these patent applications and of the corresponding granted patents, in particular the teachings of the examples are incorporated by reference into the present application.

The invention also relates to a kit for detecting microorganisms, consisting of the combination of a container having a colored bottom with a culture medium preferably containing chromogenic agents releasing chromophores in the presence of said target microorganisms.

The subject of the invention is also the use of a container having a colored bottom as a support into which is poured a culture medium preferably containing chromogenic agents releasing chromophores after the action of specific enzymes present in strains of microorganisms.

The color of the containers used (in general Petri dishes) is advantageously chosen such that it exhibits a useful contrast with the colonies of microorganisms, or the chromogens used. Useful contrast is defined as a contrast helping in the differentiation of the colors of the colonies, relative to the bottom of the dish. Thus, when the culture medium contains no chromogens, the colonies are generally light-colored and the bottom of the container is rather dark.

The color of the bottom of the dish is also chosen according to the color of the chromophores released. Thus, the blue chromogen 5-bromo-4-chloro-3-indoxyl has an absorption at 615 nm, which can be easily differentiated when red dishes are used (about 515 nm) For the chromogens producing mauve, red or purple colonies, dishes are used which have a color such that the wavelength is distant, for example yellow dishes.

When light-colored colonies are obtained, dark-colored and/or opaque dishes are used.

| | | Absorption | Dish color |
|---|---|---|---|
| Chromogen | Color | max (nm) | (example) |
| 5-Bromo-4-chloro- | blue | 615 | red |
| 3-indoxyl | | | |
| 5-Bromo-6-chloro- | red-purple | 565 | yellow |
| 3-indoxyl | (magenta) | | |
| 6-Chloro-3-indoxyl | grey-purple | 540 | opaque white, |
| | (salmon) | | or dark colored |

The chromophores which can be used for carrying out the present invention then have the following general formula, and are obtained by oxidizing a chromogenic agent linked to an enzymatic substrate. These chromophores are insoluble, inducing the formation of a colored precipitate. It should be noted that the chromophores can be obtained after the action of glycosidases, but also after the action of phosphatases.

A list of chromogenic agents may be obtained from the patent U.S. Pat. No. 6,130,057, and is incorporated by reference into the present application.

Formula of chromophores which can be used in the context of the invention:

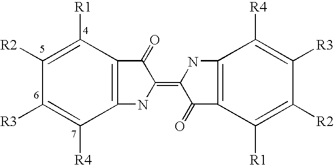

| R1 | R2 | R3 | R4 | Color |
|---|---|---|---|---|
| H | H | H | H | blue |
| Cl | Br | H | H | blue |
| H | Br | Cl | H | magenta |
| H | H | Cl | H | salmon |
| H | I | H | H | purple |
| H | Br | H | H | blue |

Another advantage of the method and of the kit according to the invention is the possibility of easily identifying the dishes comprising certain agents. Indeed, the media containing chromogenic agents are colorless before the reaction of the microorganisms and the release of the chromophores. Their packaging in colored dishes is therefore found to be useful for arranging and easily finding the media of interest, the color of the dish being chosen according to and correlated with the chromogens present in the medium, and the colors expected in the presence of microorganisms.

1. A method for detecting microorganisms present in a sample comprising the steps consisting in:
a) preparing a culture medium suitable for said target microorganisms,
b) pouring said medium into a container having a colored bottom,
c) inoculating the solid culture medium from a) with said sample or an inoculum derived from the sample and incubating,
d) detecting the presence of said microorganisms on said culture medium using the contrast between the color of the bottom of the container and the colonies isolated.

2. The method as claimed in claim 1, characterized in that said culture medium contains fluorogenic agents releasing fluorophores in the presence of said target organisms.

3. The method as claimed in claim 1, characterized in that said culture medium contains chromogenic agents appropriate for the detection of target microorganisms, by the enzymatic release of chromophores from said chromogenic agents.

4. The method as claimed in claim 1, characterized in that said container has an opaque colored bottom.

5. The method as claimed in claim 1, characterized in that said container has a transparent colored bottom.

6. The method as claimed in claim 1, characterized in that said container has a bottom having a color chosen from the colors blue, red, yellow, black or opaque white.

7. The method as claimed in claim 3, characterized in that said chromophores are released by hydrolysis by virtue of the action of an enzyme specific to the microorganism on the chromogenic agents present in the culture medium.

8. The method as claimed in claim 7, characterized in that said chromogenic agent(s) is (are) chosen from enzyme substrates linked to the metabolism of sugars, and in particular from the group consisting of
β-glucosidase substrates, β-galactosidase substrates and β-glucuronidase substrates.

9. The method as claimed in claim 3, characterized in that the said chromophore(s) is (are) chosen from the group consisting of indoxyl, haloindoxyl (bromoindoxyl, chloroindoxyl, fluoroindoxyl, iodoindoxyl, dichoroindoxyl, chlorobromoindoxyl, trichloroindoxyl), methylindoxyl and hydroxyguinoline derivatives, in particular from the following derivatives: 6-chloroindoxyl, 5-bromoindoxyl, 3-bromoindoxyl, 6-fluoroindoxyl, 5-iodoindoxyl, 4,6-dichloroindoxyl, 6,7-dichloroindoxyl, 5-bromo-4-chloroindoxyl, 5-bromo-6-chloroindoxyl, 4,6,7-trichloroindoxyl, N-methylindoxyl or 8-hydroxyquinoline.

10. The method as claimed in claim 1, characterized in that the culture medium also contains factors selective for the microorganisms being searched for.

11. The method as claimed in claim 1, characterized in that target microorganism is of the Candida species.

12. The method as claimed in claim 1, characterized in that said target microorganism is of the Staphylococcus species and said container has an opaque, preferably an opaque white, colored bottom.

13. The method as claimed in claim 1, characterized in that said target microorganism is of the Vibrio species.

14. The method as claimed in claim 1, characterized in that the said target microorganism is of the species comprising coliforms and in particular enterohemorrhagic bacteria E. coli.

15. A kit for detecting microorganisms, consisting of the combination of a container having a colored bottom and of a culture medium, the color of said container being chosen such that it exhibits a useful contrast with the color of the colonies isolated.

16. The kit as claimed in claim 15, characterized in that said culture medium contains fluorogenic agents releasing fluorophores in the presence of said target microorganisms.

17. The kit as claimed in claim 15, characterized in that said culture medium contains chromogenic agents releasing chromophores in the presence of said target microorganisms, the color of said container being chosen such that it exhibits a useful contrast with the color of the chromophores released.

18. Use of a container having a colored bottom as a support into which is poured a culture medium suitable for the growth of microorganisms, the color of the bottom of the container allowing a contrast with the colonies of said microorganisms.

19. The use as claimed in claim 18, characterized in that said culture medium contains fluorogenic agents releasing fluorophores in the presence of said target microorganisms.

20. The use as claimed in claim 18, characterized in that said culture medium contains chromogenic agents releasing chromophores after the action of specific enzymes present in strains of microorganisms, the color of said container being chosen such that it exhibits a useful contrast with the color of the chromophores released.
